(19) United States
(12) Patent Application Publication
(10) Pub. No.: US 2016/0375072 A1
(43) Pub. Date: Dec. 29, 2016

(54) CARRIER STRUCTURE IMITATING BIOLOGICAL INTERSTITIAL SYSTEM

(71) Applicants: PO-LIANG CHEN, Taipei City (TW); MA SHEN KAI RUEI CO., LTD., Tainan City (TW)

(72) Inventor: PO-LIANG CHEN, Taipei City (TW)

(21) Appl. No.: 14/823,237

(22) Filed: Aug. 11, 2015

(30) Foreign Application Priority Data
Jun. 26, 2015 (TW) 104210383

(51) Int. Cl. A61K 36/07; A61K 45/06; A61K 9/127

(52) U.S. Cl. CPC A61K 36/07; A61K 9/127; A61K 45/06

(57) ABSTRACT

Carrier structure imitating biological interstitial system is provided, comprising a carrier and a plurality of ligands. The carrier encloses particles of Taiwanofungus camphoratus. The carrier comprises first and second liposome shells. A first space is defined inside the first liposome shell. The second liposome shell encloses the first liposome shell. An external radius of the first liposome shell is smaller than an internal radius of the second liposome shell, a second space is defined between an inside of the second liposome shell and an outside of the first liposome shell. The ligands follow at least one orbit surrounding the carrier. When approaching a specific cancer cell, the ligands move towards receptors thereof and combine therewith, so that the carrier compresses; when the carrier disintegrates due to compression, the particles of Taiwanofungus camphoratus are released and then scatter on the receptors to destroy the cancer cell.

### CROSS-REFERENCES TO RELATED APPLICATION

This non-provisional application claims priority under 35 U.S.C. §119(a) on Patent Application No. 104210383 filed in Taiwan, R.O.C. on Jun. 26, 2015, the entire contents of which are hereby incorporated by reference.

### BACKGROUND

Technical Field

The present disclosure relates generally to a carrier structure imitating biological interstitial system, and more generally to such carrier structure comprising a carrier including a first liposome shell and a second liposome shell, and a plurality of ligands.

Related Art

The medical term “tumor” refers to the formation of a mass by abnormal cell proliferation which even violates surrounding or distant tissue, affecting the tissue's normal physiological function. The tumors are generally determined to be benign or malignant by histopathological examination. The malignant tumors are called cancer, and cancer cells grow rapidly and invade surrounding tissue.

In recent decades, cancer has been one of the top ten causes of death of the people in Taiwan. Treatments for cancer can be divided into three categories, namely, surgery, chemotherapy, and radiation therapy. The treatment of surgical resection focuses on removal of the tissue where cancer occurs. However, surgical resection, chemotherapy, and radiation therapy are irreversible methods that will cause destruction of human's cells, tissues, and even organs. Thus, there is an urgent need to provide a method that can effective treat or prevent cancer and will not cause irreversible secondary damage to cancer patients.

Fruiting bodies of Antrodia Camphorata grow on inner walls of heartwood of a protected species of bovine camphor (Cinnamoum kanehirai Hay) in Taiwan; the periphery of the fruiting bodies of Antrodia Camphorata bent inward, the fruiting bodies of Antrodia Camphorata, being the wood decay fungi, can break down the cellulose of the wood, thereby causing the wood rot. Since wild Antrodia Camphorata grow in a mid-altitude area of dark, moist, and low temperature, especially wild Antrodia Camphorata grow slowly, the production of the fruiting bodes will take a long time. Because cattle camphor trees are protected species, their distribution is extremely rare, plus illegal mining, the number of wild Antrodia Camphorata is quite rare and Antrodia Camphorata are expensive. Thus, the Antrodia Camphorata has a reputation of “ruby of the forest.”

Antrodia Camphorata are perennial, edible, curative mushrooms. The fruiting bodies of Antrodia Camphorata are sessile, and corky to woody, with a strong camphor aroma, and various shapes, such as plate-like, bell-shaped, or horseshoe-shaped tower, and are tightly affixed to the wood surface. The surface of the mushrooms is porous. The mushrooms grow at the beginning in seasonal red, and gradually becomes milky white, reddish brown, light brown, or pale yellowish brown. The periphery of Antrodia Camphorata often grows radially, and expands around, showing a semicircular or irregular shape.

Antrodia Camphorata, also known as Zhang Zhi or stout camphor mushroom, in taxonomy, belong to, Kingdom Fungi, Phylum Basidiomycota, Subphylum Basidiomycotina, Class Homobasidiomycetes, Order Aphyllophorales, Family Polyporaceae, Genus Antrodia.

Studies in Phytochemistry show that fruiting bodies of Antrodia Camphorata contain polysaccharide (30-50%), triterpenoids (30%), steroids, superoxide dismutase, and amino acids. In medicine, Fruiting bodies of Antrodia Camphorata have been identified as having a specific pharmaceutical effect; for example, orally administering powder made from finely grinding the fruiting bodies of Antrodia Camphorata, or extracts after boiling and drying the fruiting bodies of Antrodia Camphorata, has the effects of detoxification, anti-inflammatory, anti-tumor, inhibition of angiogenesis, and treatment of liver disease activity.

In general, the currently available health food containing the extracts of Antrodia Camphorata can be divided into tablets, capsule and the like, in terms of form. Since triterpenoids of the Antrodia Camphorata extract have a special bitter taste, the tablets of Antrodia may be more difficult to be accepted by the users, and sometimes are even refused to take by the users. For this reason, the available health food of Antrodia Camphorata products packaged in a capsule-type and filled with the compound of Antrodia Camphorata are easier to be popularized.

However, since the capsule body is mostly constructed from combining two capsule shells of smooth surface, the capsule shells at the junction may not tightly combined so that the capsule shells may loose and the Antrodia Camphorata compound inside the capsule may be scattered the outside. In addition, since the capsule itself is not a bionic structure, even after taking, the efficiency of absorption for the body is still too low.

With bionics in deep development, the human beings study and learn the organism's own internal organization and operating mode, in addition to the appearance, functions, and external mechanism. In order to provide a paradigm of “good design” for human beings, “bionic structure” is created by bionic manufacturing. In medicine, biology scientific community, the scientists draw more attention to such systems having a bionic structure: liposomes having an internal cavity that can carry drugs or other substances of the genetic structure, or carrier structures that imitates interstitial system.

For example, the use of characteristics that liposomes can be integrated with cell membranes, in order to load the material into the interior of the cell, and a biofilm imitation assembly structure with a polymer micelle structure of imitating the viral envelope, which are the focus of research in recent years. However, from a biophysical perspective, although liposomes are ideal bionic structure structural model of the imitation of cell, there are problems of poor controllability of a liposome, low mechanical strength of the liposome, low chemical stability of the liposome, and inconvenience in actual usage. In addition to the drawback that the large sizes of the particles of the bionic structure cannot meet the requirement of digestion in a short time period, transportation and efficacy are limited since the contents are not distributed specifically inside the body so that the purpose of targeting and concentration for specific regions cannot be achieved.

Thus, for the industry, there is an urgent need to develop a structure body that imitates cells not only to solve the problems of the capsule structure, tablets not easily for taken, and inefficient absorption by the body, and is available to users on easily intaking sufficient amount of Antrodia Camphorata ingredients for treatment or prevention, and has uniformity in a good size, stability of mechanics and chemistry, small particles, and facilitate digestion and absorption, and distribution in the body for specifically focusing on specific regions, and can improve the transportability and pharmacodynamics.

### SUMMARY

Accordingly, an embodiment of the invention provides a carrier structure imitating biological interstitial system. The carrier structure comprises: a carrier including a first liposome shell and a second liposome shell; and a plurality of ligands, the inside of which encloses at least a plurality of particles of Taiwanofungus camphoratus (or called Antrodia Camphorata). The carrier structure, in addition to resolving the problems as mentioned above, facilitates digestion and absorption, and distribution in the body for specifically focusing on specific regions, and can improve the transportability and pharmacodynamics.

According to an embodiment of the invention, a carrier structure imitating biological interstitial system is provided. The carrier structure comprises a carrier and a plurality of ligands. The carrier encloses and carrying a plurality of particles of Taiwanofungus camphoratus. The carrier comprises: a first liposome shell and a second liposome shell. A first space is defined in an inside of the first liposome shell. The second liposome shell encloses the first liposome shell, wherein an external radius of the first liposome shell is smaller than an internal radius of the second liposome shell, a second space is defined between the inside of the second liposome shell and the outside of the first liposome shell. The plurality of ligands follow at least one orbit surrounding the carrier. When the ligands of the carrier are approaching a specific kind of cancer cell, the ligands move towards receptors of the cancer cell and combine therewith, so that the structure of the carrier compresses; when the carrier disintegrates due to compression, the particles of Taiwanofungus camphoratus are released and then scatter on the receptors of the cancer cell so that the cancer cell is destroyed.

According to another embodiment of the invention, the carrier structure imitating biological interstitial system is provided, wherein the first liposome shell and the second liposome shell respectively comprise ethosome, the ethosome comprises a plurality of phospholipid molecules and a plurality of ethanol molecules; and the carrier the primary ingredient of the carrier comprises Phosphatidylcholine (PC), Phosphatidylserine (PS) and phosphatidylinositol (PI), and the carrier is capable of enclosing and carrying a plurality of micronized particles of Taiwanofungus camphoratus. The carrier comprises: a first liposome shell and a second liposome shell. The second liposome shell encloses the first liposome shell, wherein an external radius of the first liposome shell is smaller than an internal radius of the second liposome shell. A first space is defined in an inside of the first liposome shell. a second space is defined between the inside of the second liposome shell and the outside of the first liposome shell. The plurality of ligands follow at least one orbit surrounding the carrier.

According to another embodiment of the invention, the carrier structure imitating biological interstitial system is provided, wherein the first space is for accommodating at least a first active substance, and the second space is for accommodating at least a second active substance.

For better understanding of the above and other aspects of the invention, a plurality of embodiments or examples will be taken with accompanying drawings to provide detailed description as follows.

### BRIEF DESCRIPTION OF THE DRAWINGS

FIG. 1 is a structural schematic diagram illustrating an embodiment of a carrier structure imitating biological interstitial system, according to the invention.

FIG. 2 is a schematic diagram illustrating the operation of the carrier structure imitating biological interstitial system, according to the invention.

FIG. 3 is a schematic diagram illustrating the compression phenomenon of the carrier structure imitating biological interstitial system, according to the invention.

FIG. 4 is a schematic diagram illustrating the operation after the compression and disintegration of the carrier structure imitating biological interstitial system, according to the invention.

FIG. 5 is a simplified partial sectional view illustrating an embodiment of a carrier structure imitating biological interstitial system, according to the invention.

FIG. 6 is a simplified partial sectional view illustrating another embodiment of a carrier structure imitating biological interstitial system, according to the invention.

### DETAILED DESCRIPTION

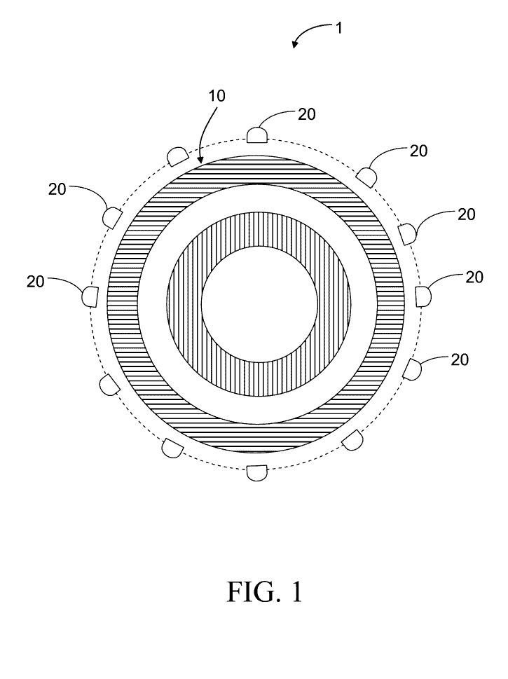

FIG. 1 is a structural schematic diagram illustrating an embodiment of a carrier structure imitating biological interstitial system, according to the invention. As shown in FIG. 1, a carrier structure 1 imitating biological interstitial system (hereinafter, carrier structure) comprises a carrier 10 and a plurality of ligands 20 (or called signal substance), wherein the ligands 20 follow at least one orbit surrounding the carrier 20.

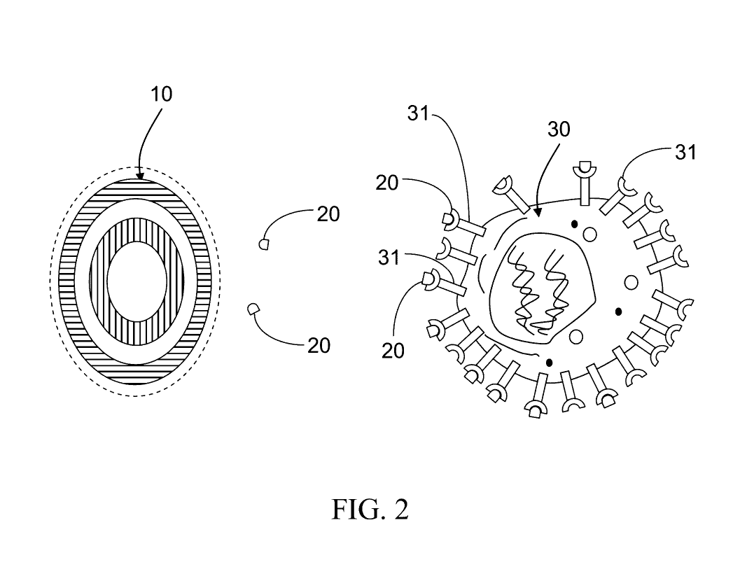

FIG. 2 is a schematic diagram illustrating the operation of the carrier structure, according to the invention. As shown in FIG. 2, when the ligands 20 of the carrier 10 are approaching a specific kind of cancer cell 30, the ligands 20 move towards receptors 31 of the cancer cell 30 and combine therewith, so that the structure of the carrier 10 compresses.

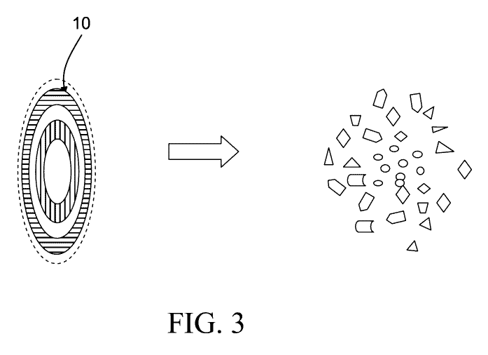

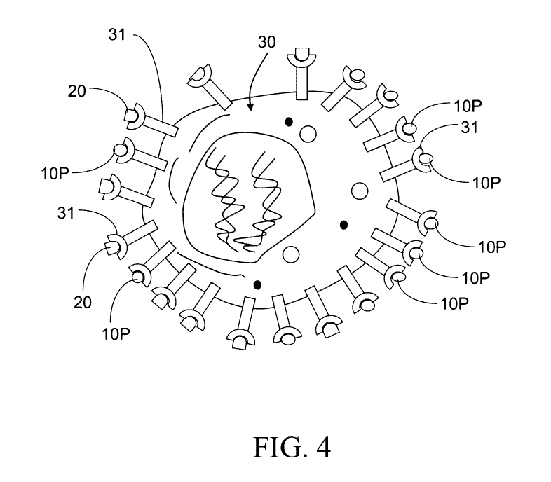

FIG. 3 is a schematic diagram illustrating the compression phenomenon of the carrier structure, according to the invention. FIG. 4 is a schematic diagram illustrating the operation after the compression and disintegration of the carrier structure, according to the invention. As shown in FIG. 3, the carrier 10 continues to compress and finally disintegrates so that ingredients 10P of the contents of the carrier 10 are released. As shown in FIG. 4, the ingredients 10P of the contents released after disintegration, e.g., the ingredients 10P of the contents inside and carried by the carrier 10, which are a plurality of particles of Taiwanofungus camphoratus; after the carrier 10 disintegrates due to compression, the particles of Taiwanofungus camphoratus are released and then scatter on the receptors 31 of the cancer cell 30 so that the cancer cell 30 is destroyed.

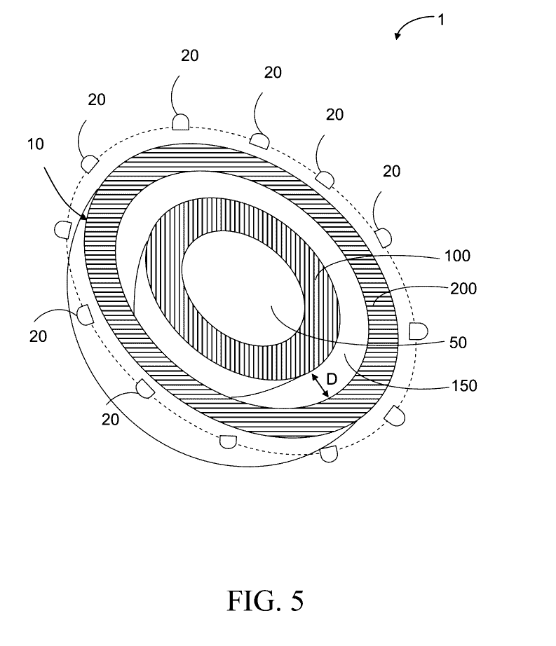

Referring to FIG. 5, a simplified partial sectional view illustrates an embodiment of a carrier structure 10, according to the invention.

According to an embodiment of the invention, the carrier 10 primarily comprises a first liposome shell 100 and a second liposome shell 200. Both first liposome shell 100 and second liposome shell are hollow spherical structured, and the first liposome shell 100 is located inside of the second liposome shell 200. A first space 50 for accommodating specific active substances is defined in the inside of first liposome shell 100. The external radius of the first liposome shell 100 is smaller than the internal radius of the second liposome shell 200, the difference between the external radius of the first liposome shell 100 and the internal radius of the second liposome shell 200 is defined as D. A second space 150 is defined between the inside of the second liposome shell 200 and the outside of the first liposome shell 100.

The first space 50 inside the first liposome shell 100 and the second space 150 between the first liposome shell 100 and the second liposome shell 200 are capable of being used to accommodate different active substances. Consequently, the carrier 10 is capable of enclosing different active substances to be transported and released. The composition of the first liposome shell 100 and second liposome shell 200 as well as the active substances accommodated in the first space 50 and second space 150 will be discussed in following paragraphs.

In addition, a plurality of ligands 20 is formed on the outside of the second liposome shell 200. Each ligand 20 comprises chitin and beta-cyclodextrin.

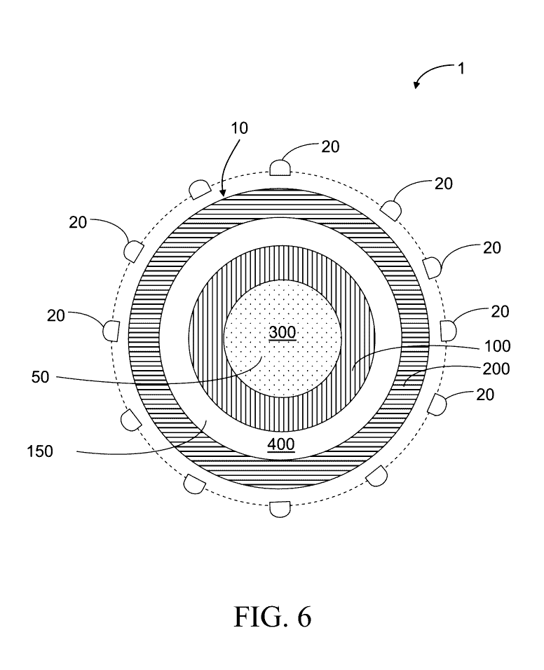

FIG. 6 is a simplified partial sectional view illustrating another embodiment of a carrier structure imitating biological interstitial system, according to the invention.

In each of the embodiments, each of the first liposome shell 100 and second liposome shell 200 is an ethosome with a hollow spherical structure. The ethosome is different from the conventional phytosome. In the ethosome, phospholipid molecules enclosed in the ethosome are not combined with the enclosed active substances. While in the conventional phytosome, the phospholipid molecules will be combined with the enclosed active substances and form new molecules, which remarkably changes the biological property of the active substances. Consequently, the ethosome is more suitable to enclose active substances having different properties than phytosome.

According to an embodiment of the invention, the carrier 10 encloses different active substances having different properties by using ethosome. The first space 50 inside the first liposome shell 100 is used for accommodating first active substances 300, and the second space 150 between the first liposome shell 100 and the second liposome shell 200 is used to enclose the second active substances 400. The second active substances 400 include a plurality of micronized particles of Taiwanofungus camphoratus (not shown), and the first active substances 300 are not exactly the same as the second active substances 400. In other words, the carrier 10 encloses the first active substances 300 by the first liposome shell 100, the second active substances 400 are enclosed between the outside of the first liposome shell 100 and the inside of the second liposome shell 200.

According to an embodiment of the invention, the carrier 10 uses the second space 150 to accommodate the micronized particles of Taiwanofungus camphoratus and other second active substances 400 to achieve the function of sustained release and targeting acting.

The first liposome shell 100 may include a plurality of phospholipid molecules of ethosome and a plurality of ethanol molecules. The second liposome shell 200 may include a plurality of phospholipid molecules of ethosome and a plurality of ethanol molecules as well.

The first active substance 300 and second active substance 400 individually are selected from a group consisted of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, micronized water-extracted and Ethanol-extracted extracts of artificially-fermented mycelia of Taiwanofungus camphoratus, and seaweed extracts, but not limited thereto. In one example, the first active substance 300 and the second active substance 400 have a plurality of different substances, and the biological properties of first active substance 300 can remain when being stored and carried.

In an embodiment, the carrier 10 is formed by basically ethosome, wherein the first liposome shell 100 and second liposome shell 200 can enclose and carry multiple active substances (i.e., the first active substances 300 and second active substances 400), and keep the first active substances 300 and second active substances 400 separated during their storage. Under utilization, the carrier 10 can be designed to release the first active substance 300 and the second active substance 400 at different locations, so that slow-release and centralized action is thus achieved.

In one embodiment, the primary ingredient of the carrier comprises Phosphatidylcholine (PC), Phosphatidylserine (PS) and phosphatidylinositol (PI), and the carrier is capable of enclosing and carrying a plurality of micronized particles of Taiwanofungus camphoratus. The previously mentioned ligands 20 can be formed on the outside of second liposome shell 200 by the combination between betacyclodextrin and Phosphatidylcholine. Be forming a plurality of ligands 20 on the outside of the second liposome shell 200, the amount of Taiwanofungus camphoratus particles being carried can be increased.

In one embodiment, the first active substance 300 and the second active substance 400 individually may be one selected from the group consisting of Antroquinonol, Antrocinnamonin A, Antroquinonol B, Antroquinonol D, Dehydroeburicoic acid, Dehydrosulphurenic acid, Zhankuic acid A, Zhankuic acid C, Antcin K, and Antcin C, or any combination thereof, but are not limited thereto.

The carrier structure according to the invention can be further modified to have the following functions:
(1) Response to external factors: for example, response to external factors of temperature, pH value, magnetic force and enzyme, so as to regulate the release of the first active substance 300 and second active substance 400 and achieve intelligent sensing and releasing.
(2) Targeting: the ethosome modified with antibody or with specific mark can make the multiple active substances be released at specified location to achieve targeting and centralized action.

To summarize, using the carrier to carry active substances of micronized Taiwanofungus camphoratus can improve the digesting, absorbing, and distributing of Taiwanofungus camphoratus which is orally taken into the human body.

It will be apparent to those skilled in the art that various modifications and variations can be made to the present disclosure without departing from the spirit and scope of the disclosure. Thus it is intended that the present disclosure cover the modifications and variations of this disclosure provided they come within the scope of the appended claims and their equivalents.

What is claimed is:

1. A carrier structure imitating biological interstitial system, the carrier structure comprising:
a carrier, for enclosing and carrying a plurality of particles of Taiwanofungus camphoratus, the carrier comprising:
a first liposome shell, wherein a first space is defined in an inside of the first liposome shell; and
a second liposome shell, enclosing the first liposome shell, wherein an external radius of the first liposome shell is smaller than an internal radius of the second liposome shell, a second space is defined between an inside of the second liposome shell and an outside of the first liposome shell; and
a plurality of ligands, following at least one orbit surrounding the carrier.

2. The carrier structure imitating biological interstitial system according to claim 1, wherein when the ligands of the carrier are approaching a specific kind of cancer cell, the ligands move towards receptors of the cancer cell and combine therewith, so that the structure of the carrier compresses; when the carrier disintegrates due to compression, the particles of Taiwanofungus camphoratus are released and then scatter on the receptors of the cancer cell so that the cancer cell is destroyed.

3. The carrier structure imitating biological interstitial system according to claim 1, wherein the first liposome shell and the second liposome shell respectively comprise ethosome, and the ethosome includes a plurality of phospholipid molecules and a plurality of ethanol molecules.

4. The carrier structure imitating biological interstitial system according to claim 1, wherein the first space is for accommodating at least a first active substance, and the second space is for accommodating at least a second active substance.

5. The carrier structure imitating biological interstitial system according to claim 4, wherein the first active substance and the second active substance are selected from the group consisting of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, micronized water-extracted and Ethanol-extracted extracts of artificially-fermented mycelia of Taiwanofungus camphoratus, and seaweed extracts, or any combination thereof.

6. The carrier structure imitating biological interstitial system according to claim 4, wherein the first active substance is not the same as the second active substance;
each of the first active substance and the second first active substance is selected from the group consisting of Antroquinonol, Antrocinnamonin A, Antroquinonol B, Antroquinonol D, Dehydroeburicoic acid, Dehydrosulphurenic acid, Zhankuic acid A, Zhankuic acid C, Antcin K, and Antcin C, or any combination thereof.

7. The carrier structure imitating biological interstitial system according to claim 3, wherein the phospholipid molecules of the first liposome shell or the second liposome shell have at least a phospholipid hydrophilic group and at least a phospholipid hydrophobic group.

8. The carrier structure imitating biological interstitial system according to claim 7, wherein the phospholipid hydrophilic group is located at the outside of the second liposome shell, the phospholipid hydrophobic group is located at the inside of the second liposome shell so as to enclose the second active substance from outside to inside.

9. The carrier structure imitating biological interstitial system according to claim 3, wherein the ethanol molecules of the first liposome shell or the second liposome shell have at least one ethanol hydroxyl and at least one hydrocarbon chain.

10. The carrier structure imitating biological interstitial system according to claim 9, wherein the ethanol hydroxyl is located at the outside of the second liposome shell, the hydrocarbon chain is located at the inside of the second liposome shell so as to enclose the second active substance from outside to inside.

11. The carrier structure imitating biological interstitial system according to claim 1, wherein the ligands comprises chitin and beta-cyclodextrin.

12. The carrier structure imitating biological interstitial system according to claim 2, wherein the first space is for accommodating at least a first active substance, and the second space is for accommodating at least a second active substance.

13. The carrier structure imitating biological interstitial system according to claim 12, wherein the first active substance and the second active substance are selected from the group consisting of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, micronized water-extracted and Ethanol-extracted extracts of artificially-fermented mycelia of Taiwanofungus camphoratus, and seaweed extracts, or any combination thereof.

14. The carrier structure imitating biological interstitial system according to claim 12, wherein the first active substance is not the same as the second active substance;
each of the first active substance and the second first active substance is selected from the group consisting of Antroquinonol, Antrocinnamonin A, Antroquinonol B, Antroquinonol D, Dehydroeburicoic acid, Dehydrosulphurenic acid, Zhankuic acid A, Zhankuic acid C, Antcin K, and Antcin C, or any combination thereof.

15. The carrier structure imitating biological interstitial system according to claim 2, wherein the first liposome shell and the second liposome shell respectively comprise ethosome, and the ethosome includes a plurality of phospholipid molecules and a plurality of ethanol molecules.

16. The carrier structure imitating biological interstitial system according to claim 15, wherein the phospholipid molecules of the first liposome shell or the second liposome shell have at least a phospholipid hydrophilic group and at least a phospholipid hydrophobic group.

17. The carrier structure imitating biological interstitial system according to claim 16, wherein the phospholipid hydrophilic group is located at the outside of the second liposome shell, the phospholipid hydrophobic group is located at the inside of the second liposome shell so as to enclose the second active substance from outside to inside.

18. The carrier structure imitating biological interstitial system according to claim 15, wherein the ethanol molecules of the first liposome shell or the second liposome shell have at least one ethanol hydroxyl and at least one hydrocarbon chain.

19. The carrier structure imitating biological interstitial system according to claim 18, wherein the ethanol hydroxyl is located at the outside of the second liposome shell, the hydrocarbon chain is located at the inside of the second liposome shell so as to enclose the second active substance from outside to inside.
